(19) United States
(12) Patent Application Publication
(10) Pub. No.: US 2025/0363665 A1
(43) Pub. Date: Nov. 27, 2025

(54) METHOD FOR DETECTING POSITION DEVIATION OF ATTACHMENT

(71) Applicant: SHANGHAI EA MEDICAL INSTRUMENTS COMPANY LIMITED, Shanghai (CN)

(72) Inventors: Mingzheng WANG, Shanghai (CN); Yang FENG, Shanghai (CN)

(21) Appl. No.: 19/124,682

(22) PCT Filed: Nov. 3, 2023
(86) PCT No.: PCT/CN2023/129747

§ 371 Date: Apr. 25, 2025

(30) Foreign Application Priority Data
Nov. 3, 2022 (CN) 202211373137.2

(51) Int. Cl. G06T 7/73; A61C 7/00; G06T 7/33; G06T 15/06; G06T 19/20; G16H 50/50

(52) U.S. Cl. CPC G06T 7/75; A61C 7/002; G06T 7/344; G06T 15/06; G06T 19/20; G16H 50/50; A61C 2007/004; G06T 2207/30036; G06T 2210/21; G06T 2210/41; G06T 2219/2004

(57) ABSTRACT

A method for detecting an attachment position deviation includes: obtaining first, second and third three-dimensional digital models, the third three-dimensional digital model representing a first attachment, the first three-dimensional digital model representing a first tooth without the first attachment, and the second three-dimensional digital model representing the first tooth with the first attachment; obtaining a target installation position of the third three-dimensional digital model on the first three-dimensional digital model; aligning the first and second three-dimensional digital models; placing the third three-dimensional digital model on the first three-dimensional digital model at the target installation position; and moving the second or third three-dimensional digital model along a surface of the third three-dimensional digital model facing the first three-dimensional digital model to align the third three-dimensional digital model with the first attachment in the second three-dimensional digital model, to obtain a deviation between an actual installation position and the target installation position.

### CROSS REFERENCE TO RELATED APPLICATIONS

The present disclosure is a national phase entry under 35 U.S.C. § 371 of International Application No. PCT/CN2023/129747 filed on Nov. 3, 2023, and claims priority of Chinese Patent Application No. 202211373137.2, filed with the China National Intellectual Property Administration (CNIPA) on Nov. 3, 2022, the entire content of which is incorporated herein by reference.

### TECHNICAL FIELD

The present application generally relates to a method for detecting whether an actual installation position of an attachment deviates from a designed target installation position.

### BACKGROUND

Due to its advantages in convenience, hygiene, and aesthetics, orthodontic treatment using shell-style orthodontic appliances made of polymer materials is becoming increasingly popular.

In some cases, in order to improve the effect of orthodontic treatment, it is necessary to place attachments on teeth and correspondingly form cavities in the shell-style orthodontic appliance to accommodate the attachments. Through the interaction between the attachments and the shell-style orthodontic appliance, the forces and/or moments required for orthodontic correction are generated.

Currently, the attachment is generally manually bonded to the tooth surface. Since the accuracy of manual operation is highly dependent on factors such as operator's experience and skills, the actual installation position of the attachment on the tooth surface may deviate from the designed target installation position, which may cause the force and/or moment applied to the tooth by the shell-style orthodontic appliance to be inconsistent with the desired force and/or moment, thereby affecting the effect of orthodontic treatment.

Therefore, it is necessary to provide a method for detecting whether the actual installation position of the attachment deviates from the designed target installation position, so that dental professionals can promptly identify and correct the position deviation of the attachment to ensure the effect of orthodontic treatment.

### SUMMARY

One aspect of the present application provides a computer-implemented method for detecting an attachment position deviation. The method includes obtaining first, second and third three-dimensional digital models. The third three-dimensional digital model is a three-dimensional digital model representing a first attachment. The first three-dimensional digital model is a three-dimensional digital model representing a first tooth without the first attachment installed. The second three-dimensional digital model is a three-dimensional digital model representing the first tooth with the first attachment actually installed. The method further includes obtaining a target installation position of the third three-dimensional digital model on the first three-dimensional digital model; performing coarse alignment between the first and second three-dimensional digital models based on a local coordinate system of the first and second three-dimensional digital models; performing fine alignment between the coarsely aligned first and second three-dimensional digital models using an ICP method; and based on a result of the fine alignment, searching along a bottom surface of the third three-dimensional digital model at the target installation position to align the third three-dimensional digital model with the first attachment in the second three-dimensional digital model, to obtain a deviation between an actual installation position of the first attachment and the target installation position.

In some embodiments, the coarse alignment is performed according to an SVD method.

In some embodiments, the coarse alignment includes: selecting a plurality of one-to-one corresponding reference points in the local coordinate system of the first and second three-dimensional digital models, each pair of reference points having the same coordinate values; and performing the coarse alignment between the first and second three-dimensional digital models based on the reference points.

In some embodiments, weights of the point pairs on which the fine alignment is based are assigned according to at least one of the following: (1) weights are assigned according to long axis coordinates of the local coordinate system: a point pair closer to an incisal edge or occlusal surface of the tooth has a higher weight, and a point pair closer to a gum line has a lower weight; (2) weights are assigned according to point pairs sorted by distance: in each iteration, point pairs are sorted from large to small by distance, and a point pair with a larger distance has a lower weight; and (3) weights are assigned according to a distance threshold: in each iteration, if the distance of a point pair exceeds a preset distance threshold, a weight of the point pair is reduced.

In some embodiments, the computer-implemented method for detecting an attachment position deviation further includes: calculating a confidence level of the fine alignment based on a proportion of point pairs that have completed the alignment.

In some embodiments, the computer-implemented method for detecting an attachment position deviation further includes: based on the coarsely aligned first and second three-dimensional digital models, casting rays along a normal direction from vertices of one of the first and second three-dimensional digital models to obtain intersection points of these rays with the other of the first and second three-dimensional digital models. The intersection points and corresponding vertices constitute a first point pair set including multiple point pairs, which serve as the point pairs on which the fine alignment is based.

In some embodiments, the second three-dimensional digital model is obtained by scanning the first tooth with the first attachment installed.

In some embodiments, the bottom surface of the third three-dimensional digital model is a plane.

In some embodiments, the computer-implemented method for detecting an attachment position deviation further includes: casting rays along the normal direction from multiple points on the bottom surface of the third three-dimensional digital model, taking intersection points of each ray with the second and third three-dimensional digital models as a point pair to constitute a second point pair set including multiple point pairs. An objective function of the searching is a sum of distances of the point pairs in the second point pair set.

In some embodiments, the computer-implemented method for detecting an attachment position deviation further includes: if a ray from a vertex on the bottom surface of the third three-dimensional digital model has no intersection point with the second three-dimensional digital model, assuming that there is a point pair on the ray, and assigning a preset distance value to the point pair as the distance of the point pair.

In some implementations, the preset distance value is greater than or equal to a maximum height of the first attachment.

On the other hand, the present application provides a computer system for detecting an attachment position deviation, which includes a storage device and a processor. The storage device stores a computer program for detecting the attachment position deviation. When the computer program is executed by the processor, the method for detecting the attachment position deviation is performed.

### BRIEF DESCRIPTION OF DRAWINGS

The above and other features of the present application will be further described below in conjunction with the accompanying drawings and detailed description thereof. It should be understood that these drawings only illustrate several exemplary embodiments according to the present application and therefore should not be considered to limit the scope of protection of the present application. Unless otherwise indicated, the drawings are not necessarily to scale and similar reference numbers represent similar components.

FIG. 1 is a schematic flow chart of a computer-implemented method for detecting an attachment position deviation according to an embodiment of the present application.

FIG. 2A schematically shows a relative positional relationship between two attachments before alignment.

FIG. 2B schematically shows a relative positional relationship between the two attachments shown in FIG. 2A after alignment.

FIG. 3 schematically shows a local coordinate system set for a three-dimensional digital model of a tooth.

FIG. 4 shows examples of determining point pairs on which the fine alignment is based.

### DETAILED DESCRIPTION

The following detailed description makes reference to the accompanying drawings, which form a part of the specification. The exemplary embodiments mentioned in the specification and drawings are for illustrative purposes only and are not intended to limit the scope of protection of the present application. In light of this application, those skilled in the art will appreciate that many other embodiments may be adopted and that various changes may be made to the described embodiments without departing from the spirit and scope of protection of this application. It should be understood that the various aspects of the present application described and illustrated herein may be arranged, replaced, combined, separated and designed in many different configurations, all of which are within the scope of protection of the present application.

One aspect of the present application provides a computer-implemented method for detecting an attachment position deviation.

Another aspect of the present application provides a computer system for detecting an attachment position deviation, which includes a storage device and a processor. The storage device stores a computer program for detecting the attachment position deviation. When the computer program is executed by the processor, the method for detecting the attachment position deviation is performed.

Currently, the most common design and manufacturing process for the shell-style orthodontic appliance is as follows. The following is a brief introduction to the process taking a single dentition (maxillary or mandibular dentition) as an example.

First, a three-dimensional digital model of the dentition under the patient's initial tooth arrangement (i.e., the patient's tooth arrangement before orthodontic treatment) is obtained by scanning. The three-dimensional digital model of the dentition can be obtained by intraoral scanning, or by scanning a physical model (e.g., a plaster model) or impression of the dentition.

Next, based on the three-dimensional digital model of the dentition under the initial tooth arrangement, a series of successive three-dimensional digital models of the dentition are generated, representing a series of successive tooth arrangements. For cases requiring attachments, the attachments are placed at selected positions of selected teeth in multiple successive three-dimensional digital models of the dentition among the series of successive three-dimensional digital models of the dentition. Hereinafter, the selected positions are referred to as the designed target installation positions.

Then, a shell-style orthodontic appliance is manufactured based on the series of successive three-dimensional digital models of the dentition. At present, the most common manufacturing method is to first use the series of successive three-dimensional digital model of the dentition to control equipment (such as stereolithography equipment) to manufacture a corresponding series of successive physical models of the dentition, and then use the hot press film molding process to press the heated film material on the series of successive physical models of the dentition to form a series of successive shell-style orthodontic appliances.

Before orthodontic treatment is performed using a shell-style orthodontic appliance with attachments, the attachments need to be installed at selected positions of selected teeth of the patient. However, as mentioned in the background, the position of the manually bonded attachment may deviate from the designed target installation position. If the deviation is too large, the effect of orthodontic treatment may be affected. Therefore, it is necessary to detect whether the installation position of the attachment deviates from the designed target installation position after the attachment is installed.

In an embodiment, the detection of whether the installation position of the attachment deviates from the designed target installation position is performed immediately after the attachment is installed.

In another embodiment, the detection of whether the installation position of the attachment deviates from the designed target installation position may also be performed when the patient returns for a follow-up visit during orthodontic treatment.

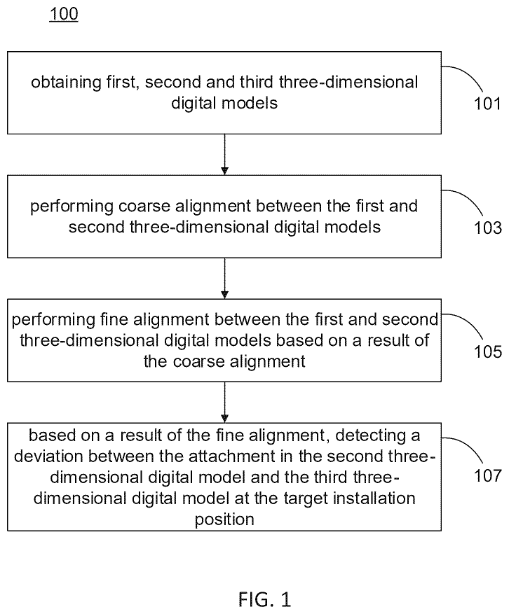

Please refer to FIG. 1, which is a schematic flow chart of a computer-implemented method 100 for detecting an attachment position deviation according to an embodiment of the present application.

In 101, first, second and third three-dimensional digital models are obtained.

The first and second three-dimensional digital models are three-dimensional digital models of the same tooth.

The first three-dimensional digital model is a three-dimensional digital model of the tooth and is obtained by scanning before the attachment is installed.

The second three-dimensional digital model is a model for which the attachment position deviation is to be detected, is a three-dimensional digital model of the tooth, and is obtained by scanning after the attachment is bonded on the tooth.

The third three-dimensional digital model is a three-dimensional digital model of the attachment.

In an embodiment, the first and third three-dimensional digital models can be three-dimensional digital models used in designing orthodontic correction plans. That is, the first and third three-dimensional digital models are in the same world coordinate system. The installation position (i.e., the designed target installation position) of the third three-dimensional digital model on the first three-dimensional digital model is known.

In 103, the first and second three-dimensional digital models are coarsely aligned.

A three-dimensional digital model of the entire dentition (maxillary or mandibular dentition) is generally obtained by scanning. In an embodiment, teeth in the three-dimensional digital model of the dentition are numbered in a predetermined manner. Teeth in two three-dimensional digital models of the same dentition can be paired based on tooth numbers, to ensure that the two three-dimensional digital models used as morphological comparison objects are three-dimensional digital models of the same tooth.

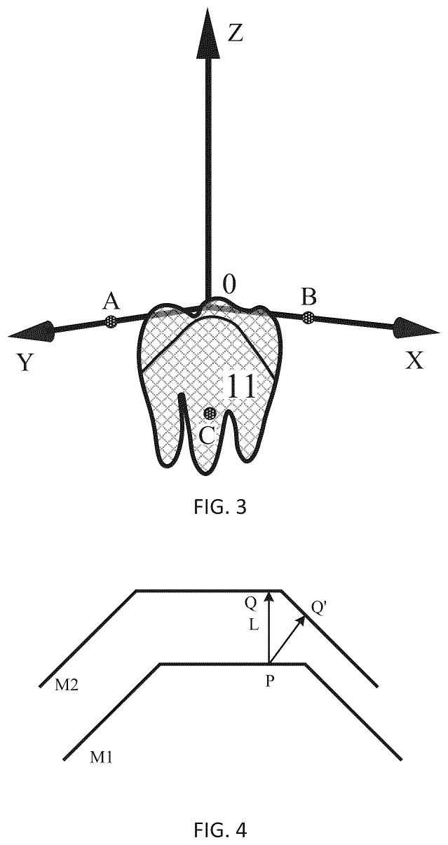

As known to those skilled in the art, in processing the three-dimensional digital model of the dentition, in order to facilitate calculation, in addition to the world coordinate system, a local coordinate system is usually set for the three-dimensional digital model of each tooth. For example, referring to FIG. 3, the origin O of the local coordinate system is located at the center of the tooth 11. The three axes (X, Y, Z) of the local coordinate system correspond to a long axis of the tooth 11, a mesiodistal direction of the tooth 11, and a labiolingual direction of the tooth 11.

The local coordinate system can be set with extremely high accuracy and consistency using current technologies (e.g., local coordinate system setting methods based on deep learning). Therefore, in an embodiment, two three-dimensional digital models of the same tooth may be coarsely aligned based on the local coordinate system.

In an embodiment, for the first and second three-dimensional digital models, at least three points on the three axes (X, Y, Z) of the local coordinate system of the first and second three-dimensional digital models may be selected as reference points. The two three-dimensional digital models of the tooth may be coarsely aligned based on these reference points. For example, four points (0, 0, 0), (1, 0, 0), (0, 1, 0), and (0, 0, 1) may be used as reference points. It can be understood that the selection of reference points is not limited to this example, as long as they are not on the same straight line. For example, as shown in FIG. 3, reference points A, B, and C are three selected reference points.

In some cases, there may be differences in the morphology of the tooth in the first and second three-dimensional digital models. For example, due to tooth wear, installation of attachment, or changes in the gum line (for example, which may be caused by the growth of erupted teeth, vertical movement or tilting of teeth, etc.).

If there are significant differences between two three-dimensional digital models of the same tooth, for example, three-dimensional digital models obtained by scanning at different time points during the tooth eruption process may have significant morphological differences, coarse alignment between the two three-dimensional digital models of the same tooth based on the local coordinate system may not work well. In such cases, feature points can be used as reference points for alignment, such as buccal cusp points, facial axis (FA) points, and proximal contact points. At present, there are many methods for identifying feature points on a three-dimensional digital model of teeth, for example, a feature point identification method based on deep learning. The identification of feature points will not be described in detail here.

In an embodiment, measurements can be made on two three-dimensional digital models of the same tooth, for example, measuring the mesiodistal width and crown height of the tooth. By comparing difference of the measurement results with a preset threshold, it can be determined whether there is a significant difference between the two three-dimensional digital models of the same tooth. If there is a significant difference, the feature points are used as reference points for coarse alignment. Otherwise, for convenience, coarse alignment can be performed based on the local coordinate system.

In an embodiment, the first and second three-dimensional digital models may be coarsely aligned based on the reference points using a Singular Value Decomposition (SVD) method.

In 105, based on the coarse alignment result, the first and second three-dimensional digital models are finely aligned.

After the coarse alignment, the first and second three-dimensional digital models are roughly aligned. On this basis, the teeth in pairs can be finely aligned.

In an embodiment, an iterative closest point algorithm (hereinafter referred to as ICP algorithm) may be used to finely align two three-dimensional digital models of the same tooth.

In an embodiment, the first and second three-dimensional digital models may be finely aligned based on a point-to-surface approach.

In an embodiment, point pairs for the fine alignment may be determined according to the following method. Some vertices sampled or all vertices selected from the first three-dimensional digital model are taken as a first point set for the fine alignment. For each point in the first point set, a ray is cast from the point along the normal direction. An intersection point of the ray with the second three-dimensional digital model (i.e., the intersection point of the ray with a surface of the second three-dimensional digital model) is obtained. The starting point of the ray and the intersection point are taken as a point pair. For example, referring to FIG. 4, for the point P selected on the first three-dimensional digital model M1, a ray L is cast from the point P along the normal direction. An intersection point Q of the ray L with the second three-dimensional digital model M2 is obtained. {P, Q} is a point pair.

Since the relative positional relationship between the first and second three-dimensional digital models is unknown, the intersection point of a unidirectional ray with the second three-dimensional digital model may not necessarily be a valid intersection point. Therefore, rays can be cast from a vertex on the first three-dimensional digital model along the normal direction in two opposite directions, or a straight line along the normal direction can be drawn passing through the vertex on the first three-dimensional digital model. In this way, two intersection points with the second three-dimensional digital model may be obtained, and the closer intersection point is selected.

In addition, the second three-dimensional digital model may lack a portion which is in the first three-dimensional digital model. Therefore, a threshold can be set. If the distance between a vertex and each corresponding intersection point is greater than the threshold, it is considered that the rays from the vertex along the normal direction have no valid intersection point with the second three-dimensional digital model.

In another embodiment, the first and second three-dimensional digital models may be finely aligned based on a point-to-point approach.

In an embodiment, point pairs for the fine alignment may be determined according to the following method. Some vertices sampled or all vertices selected from the first three-dimensional digital model are taken as a first point set for the fine alignment. For each point in the first point set, a vertex on the second three-dimensional digital model closest to the point is found, and the two vertices are taken as a point pair. For example, referring to FIG. 4, for the point P selected on the first three-dimensional digital model M1, a vertex Q′ on the second three-dimensional digital model M2 closest to the point P is found. {P, Q′} is a point pair.

In an embodiment, a first distance threshold may be set. During the iteration process, if the distance of a point pair is less than the first distance threshold, the point pair is considered to have completed the alignment. In an embodiment, the first distance threshold may be determined according to the accuracy (e.g., 0.1 mm or 0.2 mm) of a scanning device that generates the first and/or second three-dimensional digital models. For example, if the accuracy of the scanning device used is 0.1 mm, then the first distance threshold may be set to 0.08 mm, or 0.1 mm, or 0.12 mm, etc. It is understandable that the first distance threshold is not required to be equal to the scanning accuracy. Based on specific circumstances and requirements, a value within a certain range above and below the scanning accuracy can be selected as the first distance threshold.

In an embodiment, a proportion threshold may be set. If the proportion of point pairs that have completed the alignment is greater than the proportion threshold, it is considered that the fine alignment of the first and second three-dimensional digital models is completed.

In an embodiment, the following conditions can be set. If any one of these conditions is met, the iteration is stopped: (1) the proportion of point pairs that have completed the alignment is greater than the proportion threshold; (2) the number of iterations exceeds a preset iteration number threshold; and (3) the difference between the pose after this iteration and the pose after the previous iteration is less than a preset pose difference threshold (a comprehensive evaluation based on translation and rotation displacements).

Although the first and second three-dimensional digital models correspond to the same tooth, as mentioned above, due to wear, installation of attachment, and changes in the gum line, the first and second three-dimensional digital models may not completely overlap. Therefore, it is necessary to minimize the influence of these factors as much as possible during the alignment process.

In an embodiment, at least one of the following methods may be used to assign weights to the points on which the fine alignment is based, so as to minimize the influence of the above factors on the fine alignment.
(1) Weights are assigned according to long axis coordinates of the local coordinate system. A point pair closer to an incisal edge or occlusal surface of the tooth has a higher weight, while a point pair closer to a gum line has a lower weight, so as to minimize the interference caused by the change of the gum line.
(2) Weights are assigned according to point pairs sorted by distance. In each iteration, point pairs are sorted from large to small by distance, and a point pair with a larger distance has a lower weight, so as to minimize interference caused by morphological differences.
(3) Weights are assigned according to a distance threshold. The distance threshold can be set in advance. For example, the distance threshold can be set based on the scanning accuracy. In each iteration, if the distance of a point pair exceeds the distance threshold, it is considered that the distance of the point pair is caused by morphological differences, and a weight of the point pair is reduced. The weight of the point pair can even be reduced to zero, that is, the point pair does not participate in this iteration.

When the iteration of fine alignment stops, the following results are output.
(1) a rigid transformation (translation and rotation in three-dimensional space) between the first and second models.
(2) a confidence level: the proportion of point pairs that have completed the alignment. The higher the proportion, the higher the confidence level, indicating that the alignment result is more reliable and can be used as a reference for subsequent processing.
(3) an anomalous point: a point pair(s) that has/have not completed the alignment when the iteration stops. For example, the distance(s) of the point pair(s) can be compared with the above-mentioned distance threshold, and the point pair(s) with distance(s) greater than the distance threshold is/are regarded as having the morphological difference(s) between the first and second models.

Through the fine alignment, the first, second and third three-dimensional digital models are converted to the same world coordinate system. Since the installation position of the third three-dimensional digital model on the first three-dimensional digital model is known, the attachment position deviation of the second three-dimensional digital model can be detected based on the result of the fine alignment and the designed target installation position.

In 107, based on the result of the fine alignment, a deviation between the attachment in the second three-dimensional digital model and the third three-dimensional digital model at the target installation position is detected.

After the fine alignment, if the position of the attachment in the second three-dimensional digital model is not deviated, it should be aligned with the third three-dimensional digital model at the designed target installation position.

Detecting attachment position deviation essentially involves moving the third or second three-dimensional digital mode to align the two attachments. The distance and direction of the movement are the distance and direction of the deviation.

In an embodiment, the bottom surface (i.e., the installation surface) of the third three-dimensional digital model may be used as a reference surface. Some vertices sampled or all vertices selected from the reference surface are taken as a third point set. For each point in the third point set, a ray is cast from the point along its normal direction. Two intersection points of the ray with the second and third three-dimensional digital models are respectively obtained. The two intersection points on the ray are taken as a point pair. The sum of distances of all point pairs is calculated. The calculation is used as an objective function.

In an embodiment, if a ray has no intersection point with the second three-dimensional digital model, it can be assumed that a point pair exists, and a preset distance value is assigned to the point pair. In an embodiment, the preset distance value may be the maximum height of the attachment or a value greater than the maximum height of the attachment. In this way, when there is a deviation in the attachment of the second three-dimensional digital model, the result of the objective function can be relatively large.

Typically, the bottom surface of the attachment is a plane. Since the attachment position deviation is not expected to be too large, it can be approximately considered that the deviation is a deviation along the bottom surface of the attachment. Therefore, the search can be performed along directions of the bottom surface of the attachment. When moving the second or third three-dimensional digital model along four directions of the bottom surface of the third three-dimensional digital model does not reduce the result of the objective function, it is considered that the two attachments are aligned, and the moving direction and distance are used as the attachment deviation.

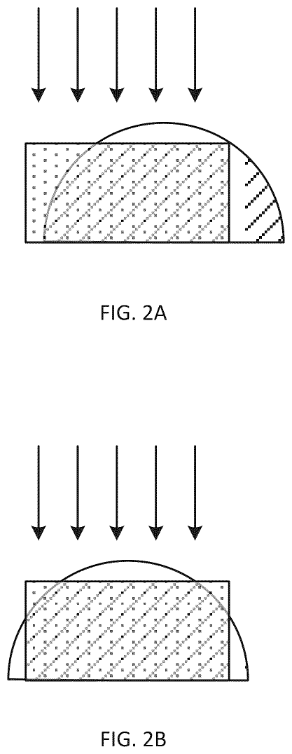

Please refer to FIG. 2A, which schematically shows the relative position relationship of the two attachments before alignment. In this figure, the rectangle represents the third three-dimensional digital model, the semicircle represents the attachment in the second three-dimensional digital model, and the arrows represent rays passing through the vertices of the bottom surface of the third three-dimensional digital model. The rectangle and semicircle in the figure do not represent the actual shapes of the attachments, but are only used to distinguish the three-dimensional digital models of the two attachments in order to facilitate the display of their positions.

Please refer to FIG. 2B, which schematically shows the relative positional relationship of the two attachments shown in FIG. 2A after alignment.

Although various aspects and embodiments of the present application are disclosed herein, other aspects and embodiments of the present application will be apparent to those skilled in the art in light of this application. The various aspects and embodiments disclosed herein are for purposes of illustration only and not limitation. The scope and essence of the present application are determined only by the appended claims.

Likewise, the various figures may illustrate exemplary architectures or other configurations of the disclosed methods and systems, which facilitate understanding of features and functionality that may be included in the disclosed methods and systems. What is claimed is not limited to the exemplary architectures or configurations shown, and the desired features may be implemented with a variety of alternative architectures and configurations. In addition, with respect to flow charts, functional descriptions, and method claims, the order of the blocks presented herein should not limit various embodiments to being implemented in the same order to perform the described functionality unless the context clearly dictates otherwise.

Unless expressly stated otherwise, terms and phrases used herein, and variations thereof, should be construed as open ended as opposed to limiting. In some embodiments, the appearance of expansive words and phrases such as “one or more,” “at least,” “but not limited to,” or other similar terms should not be understood as intending or requiring a narrowing in examples where such expansive words may not be present.

1. A computer-implemented method for detecting an attachment position deviation, comprising:
obtaining first, second and third three-dimensional digital models, wherein the third three-dimensional digital model is a three-dimensional digital model representing a first attachment, the first three-dimensional digital model is a three-dimensional digital model representing a first tooth without the first attachment installed, and the second three-dimensional digital model is a three-dimensional digital model representing the first tooth with the first attachment actually installed;
obtaining a target installation position of the third three-dimensional digital model on the first three-dimensional digital model;
performing alignment between the first and second three-dimensional digital models;
placing the third three-dimensional digital model on the first three-dimensional digital model at the target installation position; and
moving the second or third three-dimensional digital model along a surface of the third three-dimensional digital model facing the first three-dimensional digital model based on a result of the alignment to align the third three-dimensional digital model with the first attachment in the second three-dimensional digital model, to obtain a deviation between an actual installation position of the first attachment and the target installation position.

2-6. (canceled)

7. The computer-implemented method for detecting the attachment position deviation according to claim 1, wherein the second three-dimensional digital model is obtained by scanning the first tooth with the first attachment installed.

8. The computer-implemented method for detecting the attachment position deviation according to claim 1, wherein the bottom surface of the third three-dimensional digital model is a plane.

9. The computer-implemented method for detecting the attachment position deviation according to claim 1, wherein the second or third three-dimensional digital model are moved for a plurality of times, and the method further comprises:
before or after moving the second or third three-dimensional digital model for each of the plurality of times:
casting rays along a normal direction from multiple points on the surface of the third three-dimensional digital model; wherein intersection points of the rays with the second and third three-dimensional digital models are taken as point pairs which constitute a second point pair set; and
calculating a sum of distances of the point pairs in the second point pair set; and
in response to that the sum of distances does not reduce while moving the second or third three-dimensional digital model along four directions of the surface of the third three-dimensional digital model, determining that the third three-dimensional digital model is aligned with the first attachment in the second three-dimensional digital model.

10. The computer-implemented method for detecting the attachment position deviation according to claim 9, further comprising:
based on that a ray from a vertex on the bottom surface of the third three-dimensional digital model has no intersection point with the second three-dimensional digital model, assuming that there is a point pair on the ray, and assigning a preset distance value to the point pair as the distance of the point pair.

11. The computer-implemented method for detecting the attachment position deviation according to claim 10, wherein the preset distance value is greater than or equal to a maximum height of the first attachment.

12. A computer system for detecting an attachment position deviation, comprising a storage device and a processor; wherein the storage device stores a computer program for detecting the attachment position deviation, and when the computer program is executed by the processor, the method for detecting the attachment position deviation according to claim 1 is performed.

13. The computer-implemented method for detecting the attachment position deviation according to claim 1, wherein the deviation between the actual installation position of the first attachment and the target installation position is determined according to an amount of the moving.

14. The computer-implemented method for detecting the attachment position deviation according to claim 1, wherein the performing alignment between the first and second three-dimensional digital models comprises:
performing coarse alignment between the first and second three-dimensional digital models based on a local coordinate system of the first and second three-dimensional digital models; and
performing fine alignment between the first and second three-dimensional digital models after the coarse alignment using an iterative closest point (ICP) method.

15. The computer-implemented method for detecting the attachment position deviation according to claim 14, wherein the coarse alignment is performed according to a singular value decomposition (SVD) method.

16. The computer-implemented method for detecting the attachment position deviation according to claim 14, wherein the coarse alignment is performed by:
selecting a plurality of pairs of reference points in the local coordinate system of the first and second three-dimensional digital models, each pair of reference points having same coordinate values, and
performing the coarse alignment between the first and second three-dimensional digital models based on the plurality of pairs of reference points.

17. The computer-implemented method for detecting the attachment position deviation according to claim 14, wherein weights of point pairs on which the fine alignment is based are assigned according to at least one of following:
weights are assigned according to long axis coordinates of the local coordinate system: a point pair closer to an incisal edge or occlusal surface of the first tooth has a higher weight, and a point pair closer to a gum line has a lower weight;
weights are assigned according to the point pairs sorted by distance: in each iteration of the ICP method, the point pairs are sorted from large to small by distance, and a point pair with a larger distance has a lower weight; or
weights are assigned according to a preset distance threshold: in each iteration of the ICP method, based on that a distance of a point pair exceeds the preset distance threshold, a weight of the point pair is reduced.

18. The computer-implemented method for detecting the attachment position deviation according to claim 14, further comprising:
calculating a confidence level of the fine alignment based on a proportion of point pairs that complete the fine alignment.

19. The computer-implemented method for detecting the attachment position deviation according to claim 14, further comprising:
based on the first and second three-dimensional digital models after the coarse alignment, casting rays along a normal direction from vertices of one of the first and second three-dimensional digital models to obtain intersection points of the rays with the other of the first and second three-dimensional digital models;
wherein the intersection points and corresponding vertices constitute a first point pair set comprising multiple point pairs, which serve as the point pairs on which the fine alignment is based.

20. The computer-implemented method for detecting the attachment position deviation according to claim 16, wherein at least three pairs of reference points are selected, and coordinate values of the reference points are not on a same straight line.

21. The computer-implemented method for detecting the attachment position deviation according to claim 14, wherein the fine alignment is performed based point pairs using the ICP method, and during an iteration process of the ICP method, in response to that a distance of a point pair is less than a first distance threshold, the point pair completes the fine alignment;
wherein the first distance threshold is determined according to an accuracy of a scanning device that generates the first and/or second three-dimensional digital models.

22. The computer-implemented method for detecting the attachment position deviation according to claim 14, wherein the fine alignment is performed based point pairs using the ICP method, and an iteration process of the ICP method stops in response to any one or a combination of:
a proportion of point pairs that complete the fine alignment is greater than a proportion threshold;
the number of iterations exceeds a preset iteration number threshold; or
a difference between a pose of the first tooth after a current iteration and a pose of the first tooth after a previous iteration is less than a preset pose difference threshold.

23. The computer-implemented method for detecting the attachment position deviation according to claim 19, wherein:
one ray is cast along the normal direction from one vertex of the one of the first and second three-dimensional digital models to obtain one intersection point of the one ray with the other of the first and second three-dimensional digital model;
wherein the one intersection point and the vertex serve as one point pair on which the fine alignment is based.

24. The computer-implemented method for detecting the attachment position deviation according to claim 19, wherein:
two rays in two opposite directions are cast along the normal direction from one vertex of the one of the first and second three-dimensional digital models to obtain two intersection points of the two rays with the other of the first and second three-dimensional digital model; or
a straight line is drawn along the normal direction passing through one vertex of the one of the first and second three-dimensional digital models to obtain two intersection points of the straight line with the other of the first and second three-dimensional digital model;
wherein one of the two intersection points closer to the vertex than the other of the two intersection points and the vertex serve as one point pair on which the fine alignment is based.

25. The computer-implemented method for detecting the attachment position deviation according to claim 14, further comprising:
sampling multiple vertices from one of the first and second three-dimensional digital models; and
obtaining, for each vertex of the multiple vertices, a vertex on the other of the first and second three-dimensional digital models closest to the each vertex;
wherein the each vertex and the vertex obtained server as one point pair on which the fine alignment is based.
